(19) United States
(12) Patent Application Publication
(10) Pub. No.: US 2005/0256085 A1
(43) Pub. Date: Nov. 17, 2005

(54) Fungicidal mixtures

(76) Inventors: Eberhard Ammermann, Heppenheim (DE); Reinhard Stierl, Freinsheim (DE); Ulrich Schofl, Bruhl (DE); Klaus Schelberger, Gonnheim (DE); Maria Scherer, Godramstein (DE); Michael Hennigsen, Frankenthal (DE); Randall Even Gold, Obrigheim (DE)

Correspondence Address: BIRCH STEWART KOLASCH & BIRCH, PO BOX 747, FALLS CHURCH, VA, 22040-0747, US

(21) Appl. No.: 10/519,999

(22) PCT Filed: Jun. 30, 2003
(86) PCT No.: PCT/EP03/06891

§ 371 Date: Jan. 5, 2005

(30) Foreign Application Priority Data
Jul. 23, 2002 (DE) 102 33 520.6

(51) Int. Cl. A61K 31/695

(52) U.S. Cl. 514/63

(57) ABSTRACT

Fungicidal mixtures, comprising
A) at least one valinamide derivative of the formula I 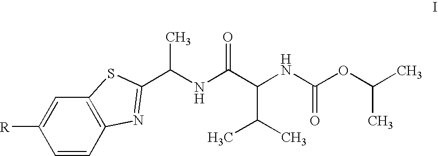 in which R is halogen, and
B) the compound of the formula II 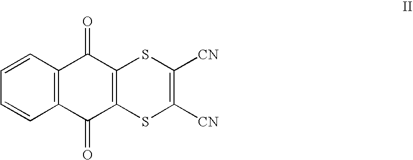 in a synergistically effective amount, methods for controlling harmful fungi using mixtures of the compounds I and II and the use of the compounds I and II for preparing such mixtures are described.

The present invention relates to fungicidal mixtures, comprising
A) at least one valinamide derivative of the formula I 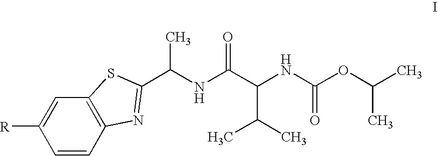
in which R is halogen, and
B) the compound of the formula II 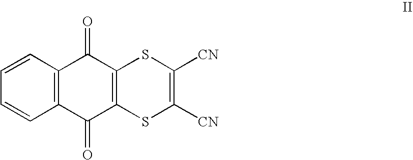
in a synergistically effective amount.

Moreover, the invention relates to methods for controlling harmful fungi using mixtures of the compounds I and II, and to the use of the compounds I and II for preparing such mixtures.

The compounds of the formula I their preparation and their action against harmful fungi are described in JP-A 09/323 984.

The compound II, (common name: dithianon) and processes for its preparation are likewise known from the literature [cf. GB-A 857 383].

Fungicidal mixtures of certain phenyl-substituted valinamides with, inter alia, dithianon are known from EP-A 610 764.

However, their action, in particular the long-term action, is frequently unsatisfactory.

It is an object of the present invention to provide mixtures which have improved activity, in particular improved long-term activity, against harmful fungi combined with a reduced total amount of active compounds applied (synergistic mixtures), with a view to reducing the application rates and widening the activity spectrum of the known compounds.

We have found that this object is achieved by the mixtures defined at the outset. Moreover, we have found that applying the compounds I and II simultaneously, i.e. together or separately, or applying the compounds I and II in succession provides better control of harmful fungi than is possible with the individual compounds alone.

In the formula I, R is a halogen atom, such as fluorine, chlorine, bromine or iodine, in particular fluorine.

The invention relates to all stereoisomers of the formula I. Preference is given to the R,S isomer.

For the mixtures according to the invention, particular preference is given to using the compound Ia (common name: benthiavalicarb).

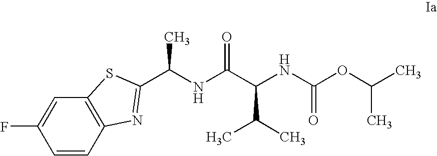

When preparing the mixtures, it is usual to employ the pure active compounds I and II, with which further active compounds against harmful fungi or other pests, such as insects, arachnids or nematodes, or else herbicidal or growth-regulating active compounds or fertilizers can be admixed as required.

The mixtures of the compounds I and II, or the simultaneous joint or separate use of the compounds I and II, have outstanding action against a wide range of phytopathogenic fungi, in particular from the classes of the Ascomycetes, Deuteromycetes, Oomycetes and Basidiomycetes. Some of them act systemically and are therefore also suitable for use as foliar- and soil-acting fungicides.

They are especially important for controlling a large number of fungi in a variety of crop plants, such as vegetable species (for example cucumbers, beans and cucurbits), fruit species, grapevine, wheat, ornamentals, sugarcane, and a variety of seeds.

They are particularly suitable for controlling the following phytopathogenic fungi: Erysiphe cichoracearum and Sphaerotheca fuliginea in cucurbits, Podosphaera leucotricha in apples, Uncinula necator in grapevines, Venturia inaequalis (scab) in apples, Septoria tritici in wheat, Botrytis cinerea (gray mold) in strawberries, vegetables, ornamentals and grapevines, Cercospora arachidicola in groundnuts, Phytophthora infestans in potatoes and tomatoes, Pseudoperonospora species in cucurbits and hops, Plasmopara viticola and Phomopsis viticula in grapevines, Alternaria species in vegetables and fruit and Fusarium and Verticillium species.

The compounds I and II can be applied simultaneously, that is either together or separately, or in succession, the sequence, in the case of separate application, generally not having any effect on the control results.

The compounds I and II are usually applied in a weight ratio of from 1:100 to 10:1, preferably from 1:20 to 5:1, in particular from 1:15 to 2:1.

Depending on the nature of the desired effect, the application rates of the mixtures according to the invention are, for the compounds I, from 5 g/ha to 500 g/ha, preferably from 50 to 500 g/ha, in particular from 50 to 200 g/ha.

Correspondingly, the application rates of the compound II are generally from 5 to 2 000 g/ha, preferably from 10 to 1 000 g/ha, in particular from 50 to 750 g/ha.

For seed treatment, the application rates of the mixture are generally from 0.001 to 1 g/kg of seed, preferably from 0.01 to 0.5 g/kg, in particular from 0.01 to 0.1 g/kg.

If phytopathogenic harmful fungi are to be controlled, the separate or joint application of the compounds I and II or of the mixtures of the compounds I and II is effected by spraying or dusting the seeds, the plants or the soils before or after sowing, or before or after plant emergence.

The fungicidal synergistic mixtures according to the invention, or the compounds I and II, can be formulated, for example, in the form of ready-to-spray solutions, powders and suspensions or in the form of highly concentrated aqueous, oily or other suspensions, dispersions, emulsions, oil dispersions, pastes, dusts, materials for broadcasting or granules, and applied by spraying, atomizing, dusting, broadcasting or watering. The use form depends on the intended purpose; in each case, it should ensure as fine and uniform a distribution as possible of the mixture according to the invention.

The formulations are prepared in a manner known per se, for example by adding solvents and/or carriers. It is usual to admix inert additives, such as emulsifiers or dispersants, with the formulations.

Suitable surfactants are the alkali metal salts, alkaline earth metal salts and ammonium salts of aromatic sulfonic acids, for example ligno-, phenol-, naphthalene- and dibutylnaphthalenesulfonic acids, and of fatty acids, alkyl- and alkylarylsulfonates, alkyl, lauryl ether and fatty alcohol sulfates, and of salts of sulfated hexa-, hepta- and octadecanols, or of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenyl ether, ethoxylated isooctyl-, octyl-, or nonylphenol, alkylphenyl polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methyl cellulose.

Powders, materials for broadcasting and dusts can be prepared by mixing or jointly grinding the compounds I and II or the mixture of the compounds I and II with a solid carrier.

Granules (for example coated granules, impregnated granules or homogeneous granules) are usually prepared for binding the active compound, or active compounds, to a solid carrier.

Fillers or solid carriers are, for example, mineral earths, such as silica gel, silicic acids, silicates, talc, kaolin, limestone, lime, chalk, bole, loess, clay, dolomite, diatomaceous earth, calcium sulfate, magnesium sulfate, magnesium oxide, ground synthetic materials, and fertilizers, such as ammonium sulfate, ammonium phosphate, ammonium nitrate, ureas and products of vegetable origin, such as cereal meal, tree bark meal, wood meal and nutshell meal, cellulose powders or other solid carriers.

The formulations generally comprise from 0.1 to 95% by weight, preferably from 0.5 to 90% by weight, of one of the compounds I and II or of the mixture of the compounds I and II. The active compounds are employed in a purity of from 90% to 100%, preferably from 95% to 100% (according to NMR spectrum or HPLC).

The compounds I and II, or the mixtures or the corresponding formulations are applied by treating the harmful fungi, or the plants, seeds, soils, areas, materials or spaces to be kept free from them with a fungicidally effective amount of the mixture, or of the compounds I and II in the case of separate application. Application can be effected before or after infection by the harmful fungi.

Examples of such preparations comprising the active compounds are:
I. a solution of 90 parts by weight of the active compounds and 10 parts by weight of N-methylpyrrolidone; this solution is suitable for use in the form of microdrops;
II. a mixture of 20 parts by weight of the active compounds, 80 parts by weight of xylene, 10 parts by weight of the adduct of 8 to 10 mol of ethylene oxide to 1 mol of oleic acid N-monoethanolamide, 5 parts by weight of the calcium salt of dodecylbenzenesulfonate, 5 parts by weight of the adduct of 40 mol of ethylene oxide and 1 mol of castor oil; a dispersion is obtained by finely distributing the solution in water;
III. an aqueous dispersion of 20 parts by weight of the active compounds, 40 parts by weight of cyclohexanone, 30 parts by weight of isobutanol, 20 parts by weight of the adduct of 40 mol of ethylene oxide and 1 mol of castor oil;
IV. an aqueous dispersion of 20 parts by weight of the active compounds, 25 parts by weight of cyclohexanol, 65 parts by weight of a mineral oil fraction of boiling point 210 to 280° C., and 10 parts by weight of the adduct of 40 mol of ethylene oxide and 1 mol of castor oil;
V. a mixture, ground in a hammer mill, of 80 parts by weight of the active compounds, 3 parts by weight of the sodium salt of diisobutylnaphthalene-1-sulfonate, 10 parts by weight of the sodium salt of a lignosulfonic acid from a sulfite waste liquor and 7 parts by weight of pulverulent silica gel; a spray mixture is obtained by finely distributing the mixture in water;
VI. an intimate mixture of 3 parts by weight of the active compounds and 97 parts by weight of finely divided kaolin; this dust comprises 3% by weight of active compound;
VII. an intimate mixture of 30 parts by weight of the active compounds, 92 parts by weight of pulverulent silica gel and 8 parts by weight of paraffin oil which had been sprayed onto the surface of this silica gel; this formulation imparts good adhesion to the active compound;
VIII. a stable aqueous dispersion of 40 parts by weight of the active compounds, 10 parts by weight of the sodium salt of a phenolsulfonic acid/urea/formaldehyde condensate, 2 parts by weight of silica gel and 48 parts by weight of water; this dispersion may be diluted further;
IX. a stable oily dispersion of 20 parts by weight of the active compounds, 2 parts by weight of the calcium salt of dodecylbenzenesulfonate, 8 parts by weight of fatty alcohol polyglycol ether, 20 parts by weight of the sodium salt of a phenolsulfonic acid/urea/formaldehyde condensate and 88 parts by weight of a paraffinic mineral oil.

The fungicidal activity of the compound and of the mixtures can be demonstrated by the following experiments:

The active compounds, separately or together, were prepared as a stock solution with 0.25% by weight of active compound in acetone or DMSO. 1% by weight of the emulsifier Uniperol® (wetting agent having emulsifying and dispersing action based on ethoxylated alkylphenols) was added to this solution, and the solution was diluted with water to the desired concentration.

Evaluation is carried out by determining the infected leaf areas in percent. These percentages are converted into efficacies.

The efficacy (E) is calculated as follows using Abbot's formula:
E=(1−α/β)·100
α corresponds to the fungal infection of the treated plants in % and
β corresponds to the fungal infection of the untreated (control) plants in %

An efficacy of 0 means that the infection level of the treated plants corresponds to that of the untreated control plants; an efficacy of 100 means that the treated plants were not infected.

The expected efficacies of the active compound mixtures are determined using Colby's formula [R. S. Colby, Weeds 15, 20-22 (1967)] and compared with the observed efficacies.

Colby's formula:
E=x+y−x·y/100
E expected efficacy, expressed in % of the untreated control, when using the mixture of the active compounds A and B at the concentrations a and b
x efficacy, expressed in % of the untreated control, when using active compound A at a concentration of a
y efficacy, expressed in % of the untreated control, when using active compound B at a concentration of b

### USE EXAMPLE 1

### Activity Against Late Blight on Tomatoes Caused by Phytophthora infestans

Leaves of potted plants of the tomato cultivar “Large Fruited St. Pierre” were sprayed to runoff point with an aqueous suspension having the concentration of active compound stated below. The next day, the leaves were infected with a cold aqueous zoospore suspension of Phytophthora infestans having a density of 0.25×10⁶ spore/ml. The plants were then placed in a water-vapor-saturated chamber at temperatures between 18 and 20° C. After 6 days, the late blight on the untreated but infected control plants had developed to such an extent that the infection could be determined visually in %.

TABLE A
Individual active compounds

| | | Concentration | |
|---|---|---|---|
| | | of active | Efficacy in % |
| | | compound in the | of the |
| | | spray liquor | untreated |
| Example | Active compound | [ppm] | control |
| 1 | Control | (90% infection) | 0 |
| | (untreated) | | |
| 2 | Ia | 3 | 89 |
| | (benthiavalicarb) | 1.5 | 83 |
| | | 0.75 | 78 |
| | | 0.375 | 67 |
| | | 0.19 | 56 |
| 3 | II | 7.5 | 0 |
| | (dithianon) | 3.75 | 0 |
| | | 1.9 | 0 |

TABLE B
Combinations according to the invention

| | Active compound | | | |
|---|---|---|---|---|
| | mixture | | | |
| | Concentration | | Observed | Calculated |
| Example | Mixing ratio | | efficacy | efficacy*) |
| 4 | Ia + II | | | |
| | 0.75 + 7.5 | ppm | 100 | 78 |
| | 1:10 | | | |
| | Ia + II | | | |
| 5 | 0.375 + 3.75 | ppm | 100 | 67 |
| | 1:10 | | | |
| | Ia + II | | | |
| 6 | 0.19 + 1.9 | ppm | 100 | 56 |
| | 1:10 | | | |
| | Ia + II | | | |
| 7 | 1.5 + 1.9 | ppm | 100 | 83 |
| | 1:1.25 | | | |
| | Ia + II | | | |
| 8 | 3 + 1.9 | ppm | 100 | 89 |

1.6:1

*)efficacy calculated using Colby's formula

### USE EXAMPLE 2

### Persistency Against Peronospora of Grapevines Caused by Plasmopara viticola

Leaves of potted grapevines of the cultivar “Müller-Thurgau” were sprayed to runoff point with an aqueous suspension having the concentration of active compound stated below. To be able to assess the persistency of the substances, the plants were, after the spraycoating had dried on, placed in a greenhouse for 3 days. Only then were the leaves inoculated with an aqueous zoospore suspension of Plasmopara viticola. The grapevines were then initially placed in a water-vapor-saturated chamber at 24° C. for 48 hours and then in a greenhouse at temperatures between 20 and 30° C. for 5 days. After this period of time, the plants were, to promote sporangia eruption, again placed in a humid chamber for 16 hours. The extent of the development of the infection on the undersides of the leaves was then determined visually.

TABLE C
Individual active compounds

| | | Concentration | |
|---|---|---|---|
| | | of active | Efficacy in % |
| | | compound in the | of the |
| | | spray liquor | untreated |
| Example | Active compound | [ppm] | control |
| 9 | Control | (96% infection) | 0 |
| | (untreated) | | |
| 10 | Ia | 3 | 84 |
| | (benthiavalicarb) | 1.5 | 79 |
| | | 0.375 | 58 |
| | | 0.19 | 48 |
| 11 | II | 3.75 | 58 |
| | (dithianon) | 1.9 | 48 |

TABLE D
Combinations according to the invention

| | Active compound | | | |
|---|---|---|---|---|
| | mixture | | | |
| | Concentration | | Observed | Calculated |
| Example | Mixing ratio | | efficacy | efficacy*) |
| 12 | Ia + II | | | |
| | 0.375 + 3.75 | ppm | 90 | 82 |
| | 1:10 | | | |
| 13 | Ia + II | | | |
| | 0.19 + 1.9 | ppm | 84 | 73 |
| | 1:10 | | | |
| | Ia + II | | | |
| 14 | 1.5 + 1.9 | ppm | 100 | 73 |
| | 1:1.25 | | | |
| | Ia + II | | | |
| 15 | 3 + 1.9 | ppm | 100 | 92 |

1.6:1.25

*) efficacy calculated using Colby's formula

The test results show that for all mixing ratios the observed efficacy is higher than the efficacy predicted using Colby's formula.

1. A fungicidal mixture, comprising
A) at least one valinamide derivative of the formula I 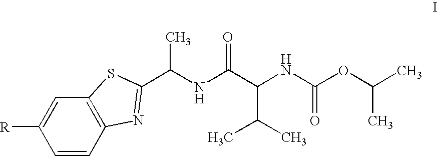
in which R is halogen, and
B) the compound of the formula II 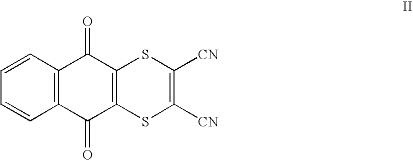
in a synergistically effective amount.

2. A fungicidal mixture as claimed in claim 1, which comprises, as valinamide derivative I, the compound Ia

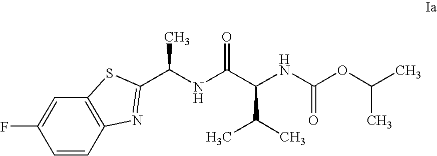

3. A fungicidal mixture as claimed in claim 1 or 2, wherein the weight ratio of the compound I to the compound II is from 10:1 to 1:100.

4. A fungicide composition comprising a solid or liquid carrier and a mixture as claimed in claim 1.

5. A method for controlling harmful fungi, which comprises treating the harmful fungi, their habitat, or the plants, seeds, soils, areas, materials or spaces to be kept free from them with a compound of the formula I and the compound of the formula II as set forth in claim 1.

6. A method as claimed in claim 5, which comprises treating the harmful fungi, their habitat, or the plants, seeds, soils, areas, materials, or spaces to be kept free from them with from 5 to 500 g/ha of the compound I as set forth in claim 1.

7. A method as claimed in claim 5, which comprises treating the harmful fungi, their habitat, or the plants, seeds, soils, areas, materials or spaces to be kept free from them with from 5 to 2000 g/ha of the compound II as set forth in claim 1.

8. The use of the compounds of the formulae I and II as set forth in claim 1 for preparing a mixture as claimed in claim 1.
